(19) United States
(12) Patent Application Publication
(10) Pub. No.: US 2005/0107680 A1
(43) Pub. Date: May 19, 2005

(54) Stereotaxic instrument with linear coordinate scales coupled to split-image microscopic image display system

(76) Inventors: J. David Kopf, Tujunga, CA (US); Daniel William Renwick, Tujunga, CA (US); Michael Milton Patterson, Plantation, FL (US)

Correspondence Address: Bruce H. Johnsonbaugh;Eckhoff & Hoppe, 333 Sacramento Street, San Francisco, CA, 94111, US

(21) Appl. No.: 10/988,825

(22) Filed: Nov. 15, 2004

Related U.S. Application Data
(60) Provisional application No. 60/523,036, filed on Nov. 18, 2003.

(51) Int. Cl. A61B 19/00; A61B 5/05; A61B 6/00

(52) U.S. Cl. 600/407; 606/130; 600/476

(57) ABSTRACT

A stereotaxic instrument is coupled to a split-screen imaging system to provide a split-screen image of the skull of the brain research subject animal along with an image of the X, Y and Z coordinates of the electrode tip (or other tool tip) being used. In addition, a third split-screen image of a recording graph displaying electrical activity recorded by an electrode may be shown. The imaging system may be a microscope/computer system or digital camera/computer system. The split-screen images may be displayed on a computer monitor or in a microscope. The system can be utilized with motor driven shift mechanisms or with manual shift mechanisms. The system allows experiments to be conducted with the subject enclosed within a Faraday cage. Remote and automatic cell searching experiments may be conducted as well.

### CROSS-REFERENCE TO RELATED APPLICATION

This application claims the benefit of and priority from U.S. provisional application Ser. No. 60/523,036 filed on Nov. 18, 2003

### BACKGROUND AND BRIEF SUMMARY OF INVENTION

The present invention relates generally to stereotaxic instruments as used, for example, in brain research conducted with animal subjects such as rats. The phrase “brain research” is used broadly to include brain, spinal cord and peripheral nerve research. These stereotaxic instruments include a manipulator capable of carrying various tools such as probes or electrodes. The position of the tool tip carried by the typical prior art manipulator in space is registered on three coordinate axes and displayed on three digital scales. In many research procedures utilizing these stereotaxic instruments, the researcher guides the placement of probes and other tools with assistance of a microscope to view the surface of the subject's skull. The researcher must continually lift his or her eyes away from the microscope to observe the digital scales which indicate the precise location of the probe or other tool relative to the skull of the research subject. The digital scales on some stereotaxic instruments are displayed on three orthogonal axes (see FIG. 7), requiring the researcher to twist and bend to read all three digital displays. A need clearly exists to simplify the task of reading digital displays while simultaneously actuating the stereotaxic manipulator and observing the procedure under a microscope.

The prior art includes a system for essentially transferring the three digital displays onto a single surface (such as a display box or computer monitor), as shown in Scouten et al U.S. patent application Publication No. US 2003/0120282 A1 dated Jun. 26, 2003. The Scouten et al device still requires a researcher using a microscope to repeatedly take his or her eyes off the microscope to read the digital displays.

The present invention solves the aforementioned problem by using split-image or split-screen technology to provide the researcher a single field of view which includes both the surface of the skull and a display of the three coordinate digital scales. The split-screen image provided by the present invention can be displayed on a computer monitor and/or in the field-of-view of a microscope.

The present invention preferably includes the stereotaxic alignment system as shown and described in the Saracione U.S. Pat. No. 6,258,103 dated Jul. 10, 2001, which is hereby incorporated by reference as though set forth in full. The '103 patent provides a stereotaxic instrument having digital scales representing the positional coordinates of a manipulator, as shown for example in FIG. 3 of that patent. The present invention is capable of functioning with stereotaxic instruments other than the '103 patent referred to above. The present invention preferably replaces the manual drive knurled knobs 214, 216 and 218 of the Saracione '103 patent with computer actuated, automatic drive motors shown and described below. The use of motorized drives for the three axes of the manipulator allows experiments to be conducted within an enclosed Faraday cage. The Faraday cage experiments reduce or eliminate unwanted electromagnetic external radiation, greatly increasing the sensitivity and accuracy of the experimental results, such as graphic recordings of brain waves, for example. In addition, the use of finer electrodes requiring greater electromagnetic isolation is facilitated by the invention.

A significant advantage of using motorized drives according to the invention is that the experimenter may be in another room from the subject in order to enable presentation of stimuli without interference. A related advantage is that experiments may be conducted “remotely” (i.e. with the experimenter in another room) if the subject is contaminated with an infectious agent or other dangerous element. In such remotely conducted experiments, the subject is preferably (but not necessarily) within a Faraday cage.

The present invention also preferably utilizes technology for producing a computer generated split-image microscopic display as shown and described in the Glaser et al U.S. Pat. No. 4,202,037 dated May 6, 1980, which patent is incorporated herein by reference as though set forth in full. The present invention is also capable of being utilized with a high resolution digital camera with a zoom lens, instead of being used with a microscope. The invention may alternately use other techniques of generating computer display “split-images” for use in creating a computer overlay in a microscope's field-of-view.

A significant aspect of the present invention is the ability to display the split-screen image on a computer monitor. The researcher can view the placement of the tool relative to the skull on the computer monitor and simultaneously observe the coordinate display on the same monitor. In this embodiment of the invention, the researcher is able to automatically change the position of a probe (or other instrument) by entering the new coordinates into the computer. The coordinates are communicated to the motorized drives for the three coordinate axes and the probe is automatically driven to the new coordinates.

Another aspect of the invention is to use the split-screen technology to also display recording graphs of brain activity sensed by the tip of an electrode.

As described below, the invention also allows, for the first time, a researcher to remotely (i.e. from another room) conduct an “automatic cell search” by combining the computer controlled drive motors for moving an electrode with the ability to monitor electrical activity as the electrode tip is moved. When electrical activity is sensed which corresponds to a pattern being sought, the automatic search ends and the electrode stops moving.

A primary object of the invention is to provide a stereotaxic instrument coupled either a split-image microscopic image display system or digital camera display system to display in a single field-of-view both an image of the experimental subject and the coordinate digital displays.

A further object is to provide a stereotaxic instrument coupled to a split screen display system which provides an image of the experimental subject and coordinate digital displays on a computer monitor and/or in the field-of-view of a microscope or digital camera.

A further object is to provide a stereotaxic instrument with motorized drives capable of being actuated under computer control.

A further object is to provide a stereotaxic instrument coupled to a split-screen computer monitor display having three images wherein the skull surface, the digital coordinates and recording graphs are all shown simultaneously in a single field-of-view.

Another object of the invention is to facilitate remote recording of brain activity as well as remote “cell searching” and automatic “cell searching” experiments.

Another object is to provide a stereotaxic instrument capable of operating automatically while being enclosed within a Faraday cage.

Other objects and advantages will become apparent from the following description and drawings wherein:

### BRIEF DESCRIPTION OF THE DRAWINGS

FIG. 1 is a schematic representation of one embodiment of the invention;

FIG. 2 illustrates a split-screen image produced in the field-of-view of the microscope of FIG. 1;

FIG. 3 illustrates the split-screen image of FIG. 2 wherein the tool tip has been moved to a different position;

FIG. 4 illustrates a split-screen image displayed on the computer monitor of FIG. 1;

FIG. 5 is a schematic representation of an alternate form of the invention from that shown in FIG. 1;

FIG. 6 is a reproduction of FIG. 3 of U.S. Pat. No. 6,258,103 and illustrates a prior art stereotaxic instrument; and

FIG. 7 is a reproduction of FIG. 4 of U.S. Pat. No. 6,258,103 and shows one form of prior art digital scale readout.

### DETAILED DESCRIPTION OF THE DRAWINGS

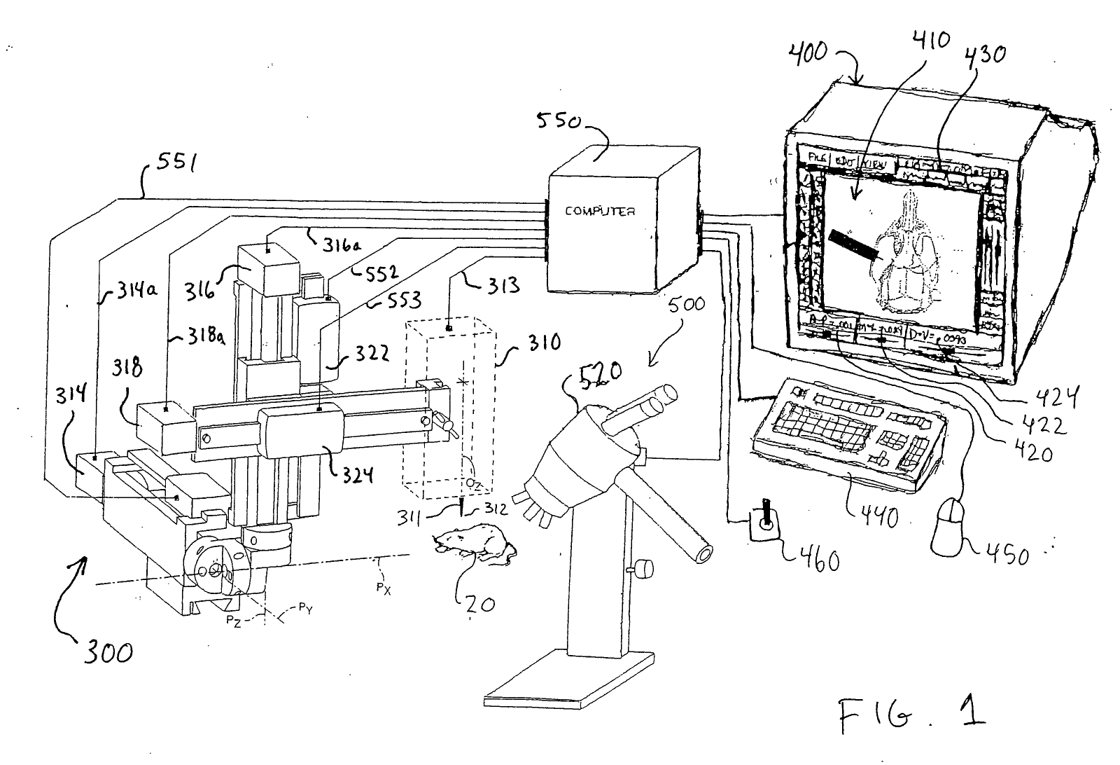

FIG. 1 is a schematic representation illustrating the present invention.

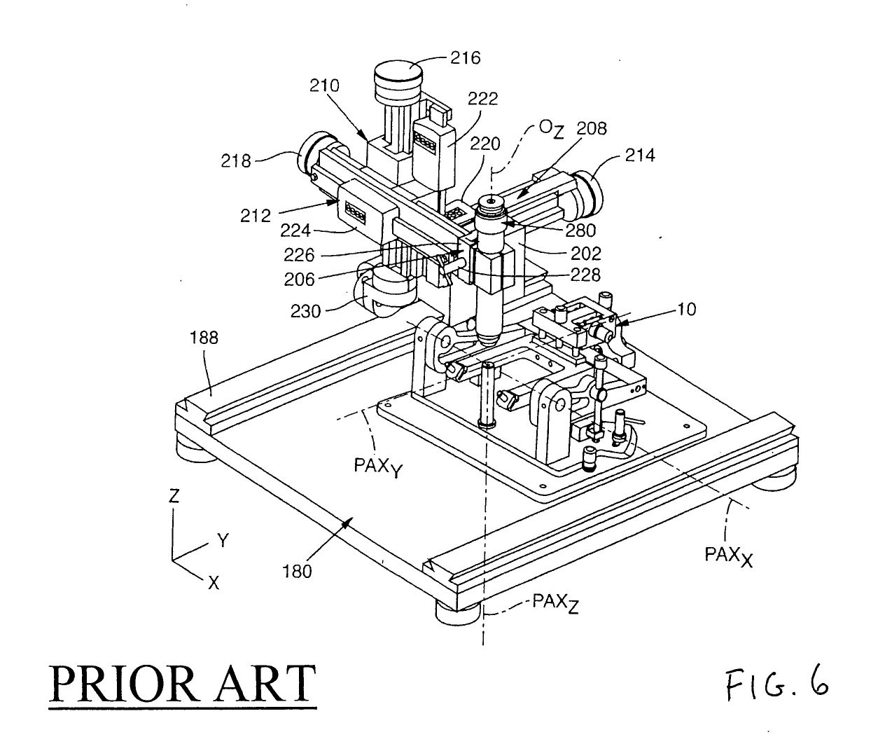

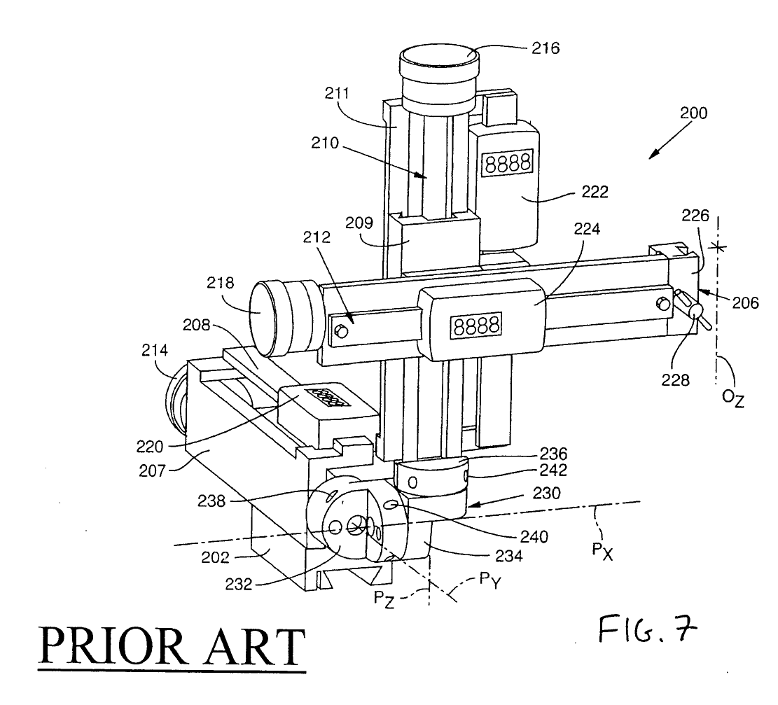

As a reference, FIGS. 6 and 7 illustrate the prior art stereotaxic instrument referred to above (U.S. Pat. No. 6,258,103) having three digital scales 220, 222 and 224 on three different orthogonally mounted carriers. The carriers include an X-shift mechanism a Y-shift mechanism and a Z-shift mechanism for moving a tool tip to selected coordinates on a Cartesian coordinate system having X, Y and Z axes. These mechanisms are described in detail in the '103 patent and are not repeated here.

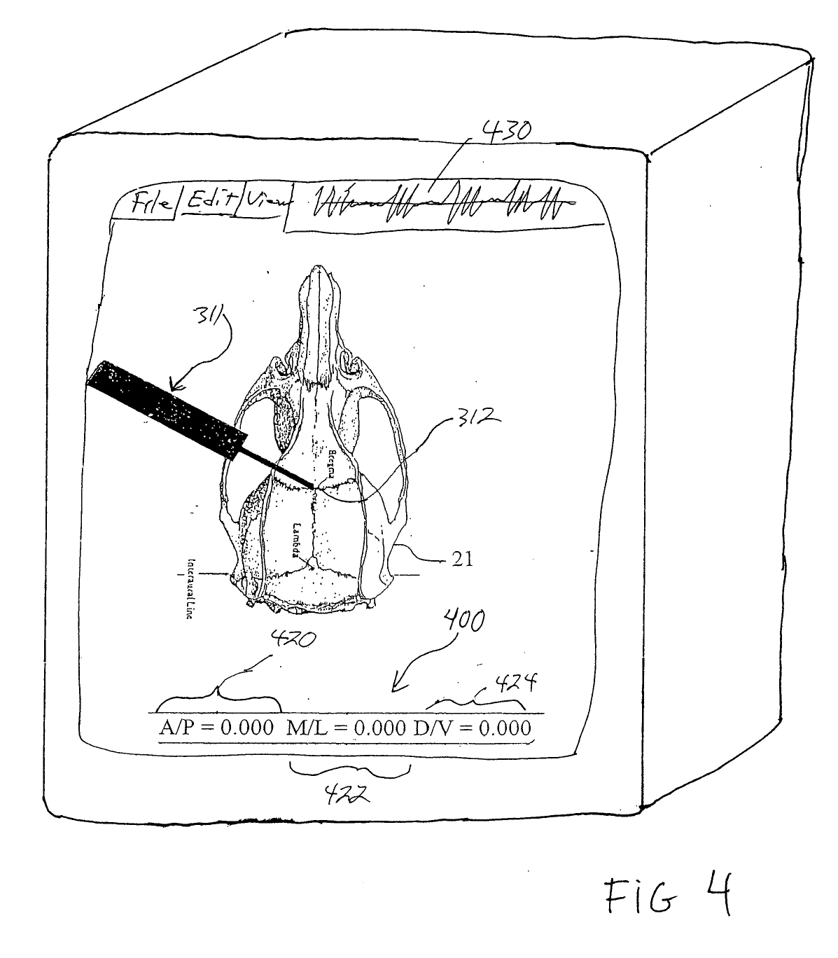

Turning back to FIG. 1, the present invention includes a manipulator 300 having three digital scales 320, 322 and 324 with readouts displayed remotely on computer monitor 400 and in the field-of-view of microscope 500. The scales 320, 322 and 324 track the movement of tool holder 310, tool 311 and tool tip 312 by tracking the movement of the X-shift, Y-shift and Z-shift mechanisms as described in the Saracione '103 patent. The manipulator 300 shown in FIG. 1 is identical to that shown and described in detail in FIG. 4 of U.S. Pat. No. 6,258,103, except that in the preferred form of the present invention, the digital scale readouts are part of the split screen display 410 on computer monitor 400 or the microscope field-of-view display 600 and the manual carrier drives 214, 216 and 218 are replaced with motors 314, 316 and 318. The detailed description of manipulator 300 is therefore not repeated here in the interest of brevity. The manipulator 300 illustrated in FIG. 1 is shown without the stereotaxic holder used in conjunction with the manipulator (for clarity). A stereotaxic holder for use with manipulator 300 is shown and described as holder 10 in FIG. 4. A detailed description of the holder 10 supporting manipulator 300 is not repeated here in the interest of brevity.

A live subject rat 20 is held by the stereotaxic holder (not shown in FIG. 1 for clarity) after having its skull aligned as shown and described in detail in U.S. Pat. No. 6,258,103.

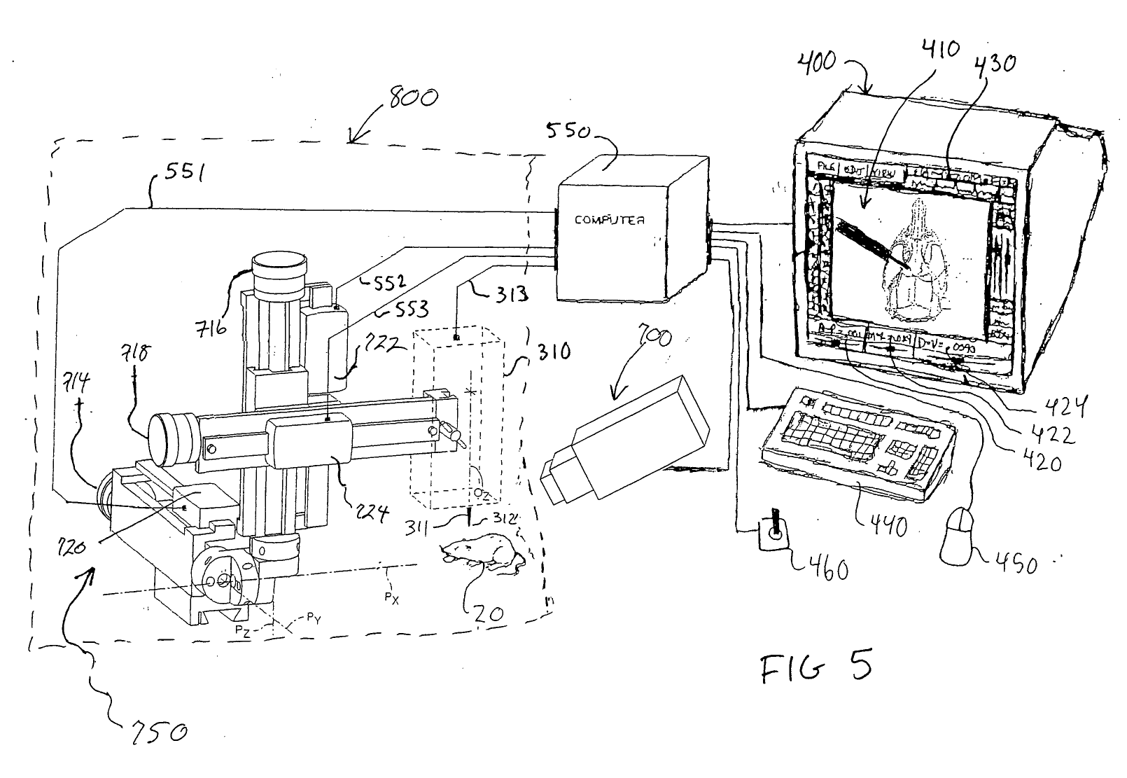

In accordance with one form of the present invention, a computer microscope system shown generally as 500 is provided which includes an optical microscope 520 and a programmable digital computer 550 having a memory for storing data. The digital programmable computer 550 receives real-time input from digital scales 320, 322 and 324 through cables 551, 552 and 553, respectively. By utilizing the split screen microscopy technique described in detail in U.S. Pat. No. 4,202,037, the readout of the digital scales 320, 322 and 324 is transferred to a computer overlay and displayed in a convenient manner in the field-of-view of optical microscope 500 and on computer monitor 400 having a display 410. It is significant to note that, instead of optical microscope 520, a digital camera may be used. The phrase “computer imaging means” as used herein and in the claims refers to either computer microscope system 500 or computer/digital camera system 700 illustrated in FIG. 5 and described below working together with computer 550.

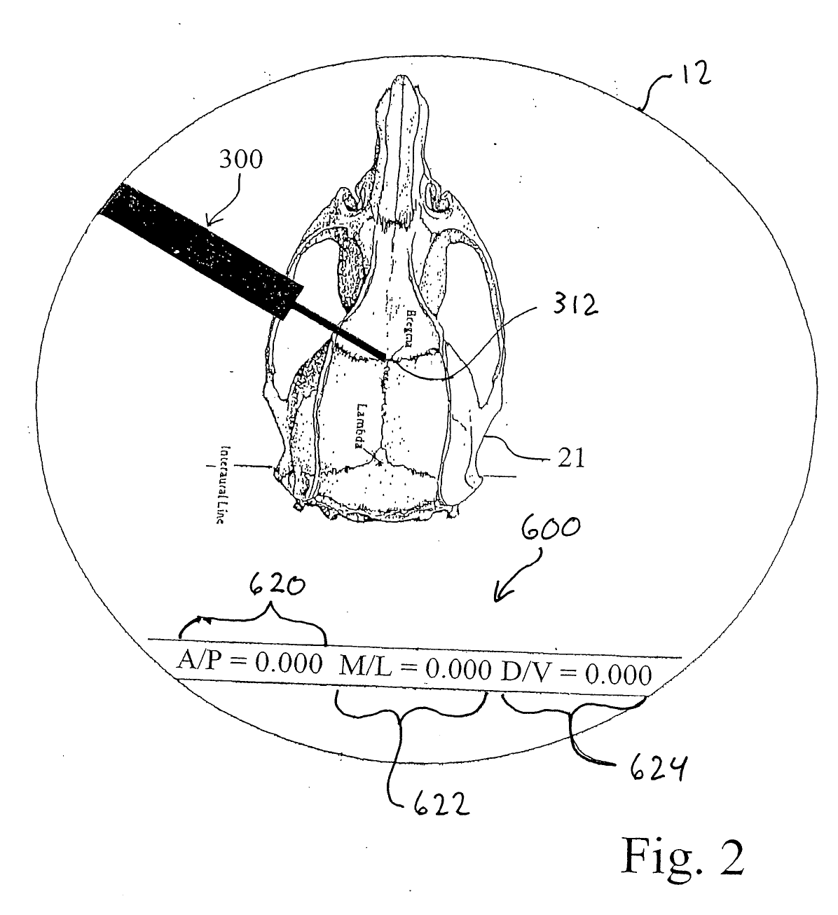

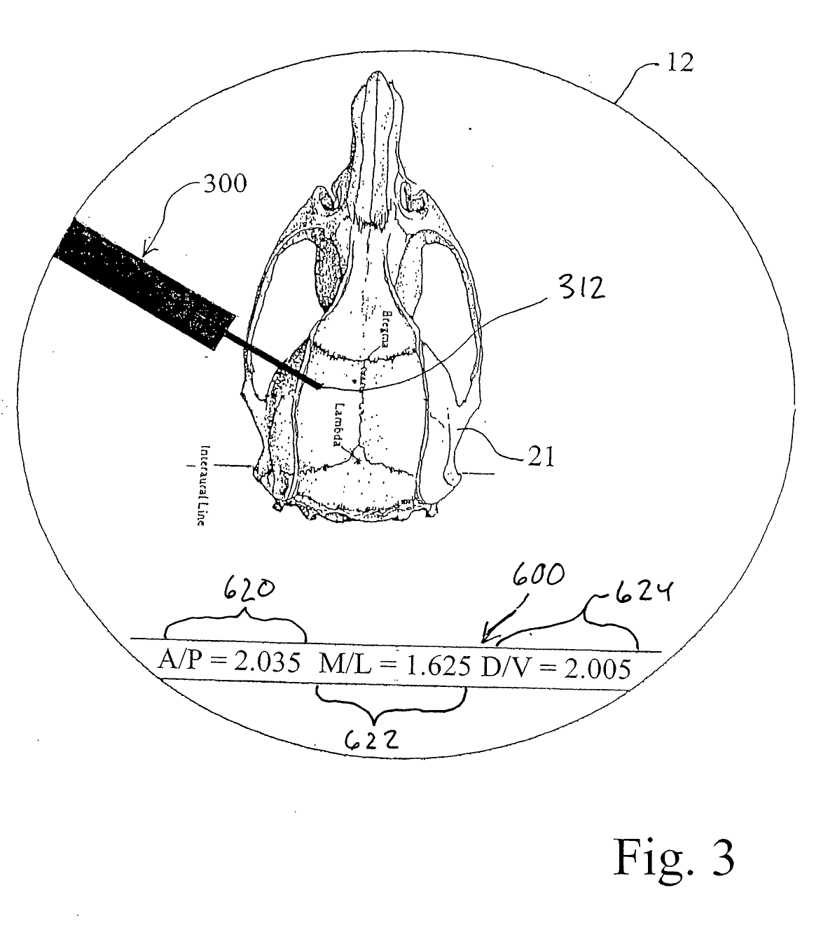

FIGS. 2 and 3 are schematic illustrations of the field of view of optical microscope 520. The field of view is circumscribed by circle 12.

To simply the following description of the invention, a skull 21 is illustrated in FIGS. 2 and 3 including illustrations of the bregma and lambda points which are typically used as reference points in brain research. In actuality, a live subject rat or other animal would be utilized and a portion of the scalp would be cut and pulled back to expose the skull sutures.

A tool 311 with tip 312 is illustrated which may be any type of tool or instrument (such as an electrode) utilized in brain research. The position of tip 312 relative to the surface of skull 21 is absolutely critical in performing many, if not all, brain research procedures.

A computer generated overlay shown generally as 600 includes three digital scale displays including “A/P” representing the anterior/posterior axis as 620. A second display shown as 622 is “M/L” representing the medial/lateral axis. A third scale 624 is shown as “D/V” which refers to dorsal/ventral which represents the depth or vertical axis. The resolution of the three scales 620, 622 and 624 may be shown in increments of one, five or ten microns.

As the researcher actuates any of the carrier drive motors 314, 316 or 318 linked to computer 550 by lines 314a, 316a and 318a (FIG. 1), the position of probe tip 312 is moved and the three digital scale readings for the position of tip 312 are automatically displayed on computer generated overlay 600 at 620, 622 and 624. The drive motors may be actuated by either a keyboard 440, mouse 450 or joystick 460. To illustrate this feature, FIG. 3 illustrates movement of tip 312 to a different position than shown in FIG. 2. In FIG. 2, the tip 312 has been moved along the medial lateral axis to a point where the tip overlies the bregma point of scalp 21. If the researcher intends to use the bregma as a reference point, the three scales are zeroed and the digital displays, as shown in FIG. 2, are all zero. As the tip 312 is caused to move by actuation of motor 318, the digital display automatically changes from that shown in FIG. 2 depending on the motion of tip 312. For example, if the tip 312 is moved laterally, posteriorly and vertically to the position shown in FIG. 3, all three displays 620, 622 and 624 will change, as shown in FIG. 3. As shown above, the researcher is able to move the tip 312 while simultaneously observing the three digital displays 620, 622 and 624 in a single field of view, for the first time in the history of stereotaxic brain research.

The carrier drive motors 314, 316 and 318 are preferably programmable, so that experiments may be repeated automatically, thereby reducing and/or eliminating human error otherwise present in such experiments.

FIG. 4 is a schematic representation of computer monitor 400 and the split-screen display provided by the invention. The central display 410 shows the skull of the subject and the tip 312 of the tool 311 (an electrode for example) being used. The digital scale displays 420, 422 and 424 are preferably located adjacent to and below the image 410 of the subject's skull. A recording graph display 430 displays electrical activity sensed by the tip of electrode 311 and transmitted on line 313 to computer 550. Display 430 is preferably located adjacent to and above the skull image. The result is a split-screen display of three images: the skull of the subject, the digital scales and the recording graph. The displays of the skull, digital scale readouts and recording graphs may be rearranged on the face of monitor 400.

FIG. 5 is a schematic representation of an alternate form of the invention from that shown in FIG. 1, utilizing a high resolution digital camera 700 preferably with a zoom lens, rather than a microscope. FIG. 5 also illustrates the use of manual, knurled knobs 714, 716 and 718 to drive the carriers on each of the X, Y and Z axes of stereotaxic instrument 750. The embodiment shown in FIG. 5 does provide the same split-screen images on monitor 400 as does the embodiment shown in FIG. 1. The digital camera 700 may also be used together with the motorized drives on each carrier as shown as items 314, 316 and 318 of FIG. 1. A Faraday cage 800 surrounds the stereotaxic instrument 750 and the animal subject 20. Faraday cage is preferably used with motorized drives 314, 316 and 318, as shown in FIG. 1, since the cage 800 need not be opened to actuate the carriers to move the tool tip, as is the case with the prior art.

The foregoing description of the invention has been presented for purposes of illustration and description and is not intended to be exhaustive or to limit the invention to the precise form disclosed. Modifications and variations are possible in light of the above teaching. The embodiments were chosen and described to best explain the principles of the invention and its practical application to thereby enable others skilled in the art to best use the invention in various embodiments and with various modifications suited to the particular use contemplated. The scope of the invention is to be defined by the following claims.

1. A stereotaxic instrument having linear digital scales for X, Y and Z axes and being coupled to a split-image microscopic or digital camera image display system, comprising:
a stereotaxic instrument for supporting an experimental subject, said instrument having a tool holder for carrying a tool with a tool tip, said instrument also having an X-shift mechanism, a Y-shift mechanism and a Z-shift mechanism for moving said tool tip to selected coordinates on a Cartesian coordinate system having X, Y and Z axes,
linear digital scales adapted to track the movement of said tool tip with respect to said X, Y and Z axes, and
computer imaging means for imaging said experimental subject either with a microscope or digital camera and being coupled to said linear digital scales for generating in a single field-of-view a split-image display of said experimental subject, said tool tip and the readout of said digital scales showing the X, Y and Z coordinates of said tool tip.

2. The apparatus of claim 1 further comprising a monitor on which said split-image is displayed.

3. The apparatus of claim 1 wherein said computer imaging means is a computer microscope and said split-image is displayed within the field-of-view of said computer microscope.

4. The apparatus of claim 1 further comprising three motors, one of which is connected to each of said X-shift mechanism, said Y-shift mechanism and said Z-shift mechanism and wherein each motor is connected to and controlled by said computer imaging means.

5. The apparatus of claim 4 further comprising a Faraday cage enclosing said stereotaxic instrument and said experimental subject.

6. The apparatus of claim 2 further comprising a recording graph split-screen image displaying electrical brain activity being sensed by an electrode and displayed on said monitor to produce in a single field-of-view the experimental subject, said X, Y and Z digital scales and a recording graph of electrical brain activity.
